(19) United States
(12) Patent Application Publication
(10) Pub. No.: US 2007/0021688 A1
(43) Pub. Date: Jan. 25, 2007

(54) Method and system for treating irritable bowel syndrome

(76) Inventor: Norman Godin, Geneva (CH)

Correspondence Address: COZEN O'CONNOR, P.C., 1900 MARKET STREET, PHILADELPHIA, PA, 19103-3508, US

(21) Appl. No.: 10/555,363

(22) PCT Filed: May 6, 2003
(86) PCT No.: PCT/US03/13773

§ 371 Date: Nov. 2, 2005

(51) Int. Cl. A61B 5/103

(52) U.S. Cl. 600/593

(57) ABSTRACT

A method and system for treating Irritable Bowel Syndrome (“IBS”) using biofeedback is disclosed. In one aspect the invention is a method comprising: inserting a probe (13) into a bowel (15) of a patient having IBS, the probe (13) being adapted to measure pressure at one more locations within the bowel (15) and obtain pressure data; transmitting the pressure data to a receiver (50) having a processor, processing the pressure data with the processor to determine an aggravated state; and signaling the aggravated state to the patient (100). The patient (100) can then be instructed to take measures to alleviate, reduce, or delay onset of the aggravated states such as performing relaxation exercises, resting, meditating, massaging the abdomen, or relating the aggravated state to ingestion of certain foods. The inventive method can use the determination of the aggravated state to send a regulation signal to a colon pace-setter positioned within the bowel (15) which will then regulate muscle contractions of the bowel (15).

### TECHNICAL FIELD OF THE INVENTION

The invention relates generally to the field of bio-feedback methods and systems, and specifically to methods and systems for treating irritable bowel syndrome using bio-feedback data.

### BACKGROUND ART

Irritable Bowel Syndrome (“IBS”) is a disorder that interferes with the normal functions of the large intestine (colon/bowel). IBS causes stomach pain, bloating, and abnormal bowel movements. People with IBS often have chronic diarrhea, constipation, or both. Some patients have no abdominal pain in the morning, and get stomachaches in the afternoon. Others go for two weeks without pain, and then have a day of crippling pain.

It is estimated that as many as one in five Americans has IBS, making it one of the most common disorders diagnosed by doctors. Generally, IBS is more common in women than in men and usually begins around age 20.

IBS causes a great deal of discomfort and distress. Some people can control or at least reduce their IBS symptoms with diet, stress management, and/or medications prescribed by their physician. However, IRS can be disabling for some people, causing them to be unable to work, go to social events, or travel even short distances. Symptoms of IBS cannot be traced to a single organic cause. As used herein, the terms bowel and colon are used interchangeably. Research suggests that people with IBS seem to have a bowel that is more sensitive and reactive than usual to a variety of things, including certain foods and stress. IBS symptoms result from the bowel not working properly. For example, the bowel can be spasmodic or can even stop temporarily. Spasms within the bowel are sudden strong muscle contractions that come and go. The lining of the bowel, the epithelium, which is affected by the immune and nervous systems, regulates the passage of fluids in and out of the bowel. In patients with IBS, the epithelium appears to work properly but fast movement of the bowel's contents can overcome its absorptive capacity.

In people with IBS, the bowel responds strongly to stimuli, such as food or stress, that would not bother most people. It has been found that stress, emotions, and diet can strongly affect the bowel's functioning and play a significant role in the onset of the pain experienced by IBS sufferers. The following have been associated with the onset of IBS symptoms: large meals; bloating from gas in the colon; medicines; wheat, rye, barley, chocolate, milk products, or alcohol; drinks with caffeine, such as coffee, tea, or colas; and stress, conflict, or emotional upsets.

Stresses, such as feeling mentally or emotionally tense, troubled, angry, or overwhelmed will affect the nerves within the bowel. These nerves control the normal rhythmic contractions of the bowel and cause abdominal discomfort at stressful times. People often experience cramps or “butterflies” when they are nervous or upset. But with IBS, the bowel can be overly responsive to even slight conflict or stress. Stress also makes the mind more tuned to the sensations that arise in the bowel and makes the stressed person perceive these sensations as unpleasant.

Bowel motility, the contraction of the bowel muscles and the movement of its contents, is controlled by nerves and hormones and by electrical activity in the bowel muscle. If the muscles of the bowel do not contract in a coordinated way, or if they contract in an irregular way or with increased force (i.e. pressure), the contents within the bowel do not move smoothly, resulting in abdominal pain, cramps, constipation or diarrhea, and/or a sense of incomplete stool movement.

No cure has been found for IBS. While medications such as alosetron hydrochloride (“Lotronex”), tegaserod maleate (“Zelnorm”), laxatives, and antidepressants are used to treat IBS, these medications are not effective in all people and do not entirely prevent the onset of IBS symptoms. Moreover, some of these medications have serious side effects, such as severe constipation or decreased blood flow to the bowel. Laxatives can be habit forming if they are used frequently. Thus, a need still exists for ways to treat patients with IBS to alleviate their symptoms and help them live normal lives.

As mentioned above, stress and diet have been linked to the onset of IBS symptoms, particularly the increased or irregular pressure patterns in the bowel that result from bowel muscle spasms/contractions. When the bowel is functioning normally, it produces orderly, gentle muscular contractions, alternating with relaxation, that propel its contents toward the rectum and anus for elimination. In patients with 113S, the muscular contractions are disorganized, harsh, or spasmodic. These increased or irregular pressure patterns in the bowel can cause abdominal pain, feelings of discomfort, and irregular bowel movement. If a person can detect when there are increased or irregular pressure patterns in the bowel, he/she may take certain measures to prevent, delay, or reduce the onset of painful or inopportune IBS symptoms.

Stress management and diet control/awareness are important part in treating IBS. It has been found that stress reduction (relaxation) training, relaxation therapies, can help mediate IBS symptoms. However, no satisfactory method or apparatus exits to help people suffering from IBS to: (1) detect when their bowels are in an irregular or increased pressure state (i.e. an aggravated state”); and (2) signal the existence of the aggravated state to the patient so he/she may take the appropriate remedial actions in time.

Currently, systems exist that are capable of monitoring and recording pressure within an internal cavity of a patient's body and transmitting the retrieved data to an externally located receiver. One such system is disclosed in U.S. Pat. No. 6,285,897, Kilcoyne et al., the teachings of which are incorporated herein by reference. While Kilcoyne discloses the concept of placing a wireless monitor within an internal cavity that sends biofeedback data to an externally located receiver for analysis, the concept of using the system as a means to treat IBS is not disclosed. Additionally, the disclosed system does not teach methods or apparatus that will process the incoming biofeedback data and immediately signal the patient of a significant change in the data readings. Thus, a need still exists for a system and method of treating IBS that will obtain biofeedback data within a patient's bowel and alert the patient of an aggravated pressure state so that remedial action may be taken.

Solid state manometry probes and water perfused manometry probes exist are designed to take pressure measurements within hollow organs. While these manometry apparatus exist, no one has ever used these probes in a way to solve the above need.

### DISCLOSURE OF THE INVENTION

It is an object of the present invention to provide a system and method for treating IBS.

Another object is to provide a system and method for treating IBS that informs a patient of an aggravated state in his/her bowel in sufficient time so that he/she can take remedial action.

Yet another object is to provide a system and method for treating IBS that informs a patient of an aggravated state in his/her bowel so that he/she can realize that their discomfort is from IBS.

Still another object is to provide a system and method for treating IBS that informs a patient of an aggravated state in his/her bowel so that he/she can identify the source of stress and/or dietary intake causing IBS symptoms.

A further object is to provide a system and method for treating IBS that informs a patient of an aggravated state in his/her bowel so that he/she can recognize patterns in their IBS symptoms.

Another object is to provide a system and method for treating IBS that informs a patient of an aggravated state in his/her bowel in sufficient time so that remedial action can be taken.

A still further object is to provide a system and method for treating IBS that gives a patient confidence to partake in normal social activities.

These and other objects are met by the present invention which in one aspect is a method for treating IBS comprising: inserting a probe into a bowel of a patient having IBS, the probe adapted to measure pressure at one more locations within the bowel and obtain pressure data; transmitting the pressure data to a receiver having a processor; processing the pressure data with the processor to determine an aggravated state; and signaling the aggravated state to the patient. Preferably, the method further comprises the step of instructing the patient to take measures to alleviate, reduce, or delay onset of the aggravated state. Such measures can include performing relaxation exercises, resting, meditating, massaging an abdomen of the patient, and relating the aggravated state to ingestion of certain foods.

The method of the present invention is preferably performed by affixing either a solid state manometry probe or a water perfused manometry probe to a wall of the bowel. The probe can be affixed used any known method in the art, such as gluing, suturing, stapling, or using tacks, pins, elastic bands, or other attachments that at least partially capture or penetrate the wall of the bowel. The probe, once affixed, preferably transmits the pressure data it obtains to the receiver via wireless means, such as a radio frequency transmitter. Alternatively, the pressure data obtained by the probe can be transmitted to the receiver via a wire within a catheter.

The receiver can signal the patient of the presence of the aggravated state by creating an audio signal through a speaker, displaying an image on a display screen/area, illuminating a light source such as an LED, vibrating, or displaying the measured pressure data on the receiver.

In another aspect, the invention is a system for treating IBS comprising: a pressure probe adapted to be inserted into a bowel of a patient having IBS, the pressure probe having means to measure pressure at one more locations within the bowel and obtain pressure data and means to transmit the pressure data; a receiver having means to receive the pressure data transmitted by the pressure robe; a processor programmed to process the pressure data to determine an aggravated state; and means to signal the aggravated state to the patient. The system preferably has means to instruct the patient to take measures to alleviate, reduce, or delay onset of the aggravated state. The means to instruct the patient can be an image on a display area of the receiver. The measure can include instructing the patient to perform relaxation exercises, rest, meditate, or avoid foods causing increased bowel pressure.

The means to signal the patient of the aggravated state can be a speaker, an image display, a light source, a vibration source, or a pressure display. It is further preferable that the means to signal be located on the receiver. The pressure probe can be a solid state manometry probe or a water perfused manometry probe.

It is further preferred that the pressure probe be wireless and adapted to be fixed to a wall of the bowel. In this embodiment wireless technology is used and the means to transmit the pressure data can be a radio frequency transmitter and the means to receive the pressure data can be a radio frequency receiver. Alternatively, the means to transmit the pressure data can be a wire within a catheter. Finally, it is preferred that the processor and the means to signal to be located within the receiver.

In yet another aspect, the invention is a method for treating IBS comprising: inserting a probe into a bowel of a patient having IBS, the probe adapted to measure pressure at one more locations within the bowel and obtain pressure data; inserting a colon pace-setter into the bowel, the colon pace-setter adapted to regulate muscle contractions of the bowel upon receipt of a regulation signal; transmitting the pressure data to a receiver having a processor; processing the pressure data with the processor to determine an aggravated state; upon determination of an aggravated state, generating the regulation signal with the processor; and transmitting the regulation signal to the colon pace-setter. The colon pace-setter and the probe can be contained within a single device. The regulation signal is preferably transmitted from the receiver to the colon-pace setter via wireless means.

### BRIEF DESCRIPTION OF DRAWINGS

FIG. 1 is a side view of a section of a colonoscope coupled to a delivery cable positioning a solid state manometry probe to the wall of a bowel according to the present invention.

FIG. 2 is a schematic of the solid state manometry probe of FIG. 1 communicating via radio frequency with a receiver according to the present invention.

FIG. 3 is a perspective view of the receiver shown in FIG. 2.

FIG. 4 is a schematic of the electronics within the receiver of FIG. 3.

FIG. 5 is a flowchart of an embodiment of a method of treating IBS according to the present invention.

### MODES FOR CARRYING OUT THE INVENTION

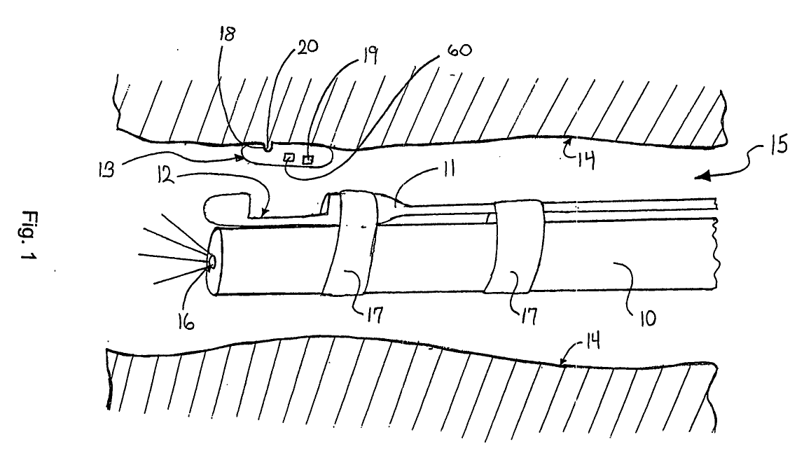

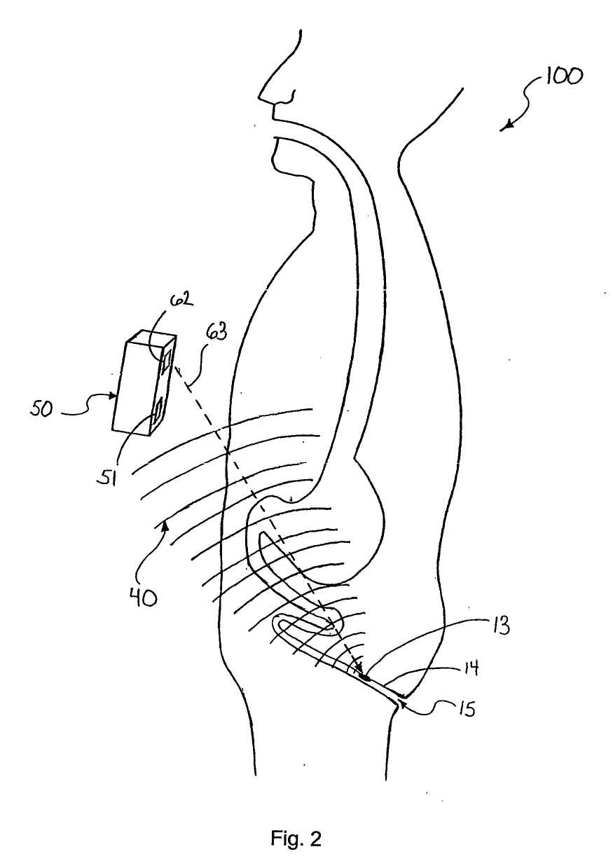

Referring to FIG. 1, colonoscope 10 is coupled to delivery cable 11. Delivery cable 11 comprises niche 12 and colonoscope light 16. Tape 17 is used to connect colonoscope 10 to delivery cable 11 for insertion into the bowel cavity 15 of a patient 100 (FIG. 2) having IBS. Niche 12 is adapted to receive solid-state manometry probe 13. Niche 12 holds solid-state manometry probe 13 during the insertion of colonoscope 10 and delivery cable 11 into bowel cavity 15. Colonoscope light 16 provides illumination so that solid-state manometry probe 13 can be properly positioned on bowel/mucosoal wall 14 of bowel cavity 15.

Solid state manometry probe 13 comprises aspiration orifice 18 and radio frequency transmitter 19. Colonoscope 10 and delivery cable 11 are fed into bowel cavity 15 until the position is reached where it is desirable to affix solid-state manometry probe 13 to bowel wall 14 for monitoring the bowel wall 14 and bowel cavity 15 for irregular or increased pressure fluctuations caused by muscle contractions related to IBS. Once the proper position is reached, colonoscope 10 and delivery cable 11 are manipulated so that solid-state manometry probe 13 contacts the bowel wall 14 of bowel cavity 15. Aspiration orifice 18 is then activated, pulling in a mucosoal portion 20 of the bowel wall 14 of bowel cavity 15. Once aspiration orifice 18 is filled with portion 20 of bowel wall 14, a barb 21 within solid state manometry probe 13 is activated, piercing portion 20 and securing solid state manometry probe 13 securely to bowel wall 14.

Solid-state manometry probe 13 is adapted to measure pressure within bowel cavity 15 and can detect changes in pressure during stimulation (i.e., muscle contraction). Solid-state manometry probe 13 preferably has several measurement ports spaced at defined intervals (not illustrated). Each measurement port comprises a miniaturized solid state transducer that can measure and detect changes in pressure. Thus, the coordination of contractile activity along wall 14 of bowel 15 can be measured as well as pressure in the bowel cavity 15.

Alternatively, a water-perfused manometry probe can be used in replace of a solid-state manometry probe. Water-perfused manometry probes rely on an assembly of thin, water-perfused tubes with side holes spaced a various distances. These thin catheters are glued together forming a tube. To record pressure changes, the catheter is connected to a low compliance multi-channel perfusion system. The low compliance of both the perfusion system as well as the catheter is critical to minimize measurement artifacts as high compliance would blunt the amplitude of pressure change and significantly limit the rate at which such changes can be properly recorded. However, a solid-state manometry probe is preferred because they are thinner and respond more rapidly to pressure changes and therefore more accurately reflect rapid changes in pressures, such as the relaxation or contraction of the bowel muscles.

Referring now to FIG.2, solid-state manometry probe 13 is properly placed in the bowel 15 of IBS patient 100. Solid-state manometry probe 13 is in wireless communication with receiver 50 via RF signals 40. Such communication technology is known in the art and is disclosed in U.S. Pat. No. 6,285,897, which is incorporated herein by reference in its entirety. Through the use of radio frequency (“RF”) transmitter 21 (FIG. 1), solid-state manometry probe 13 transmits the collected pressure data to receiver 50 (FIG. 2) as RF signals 40 for processing. Receiver 50 comprises an RF receiver 51 that is adapted to receive RF signals 40 from solid-state manometry probe 13.

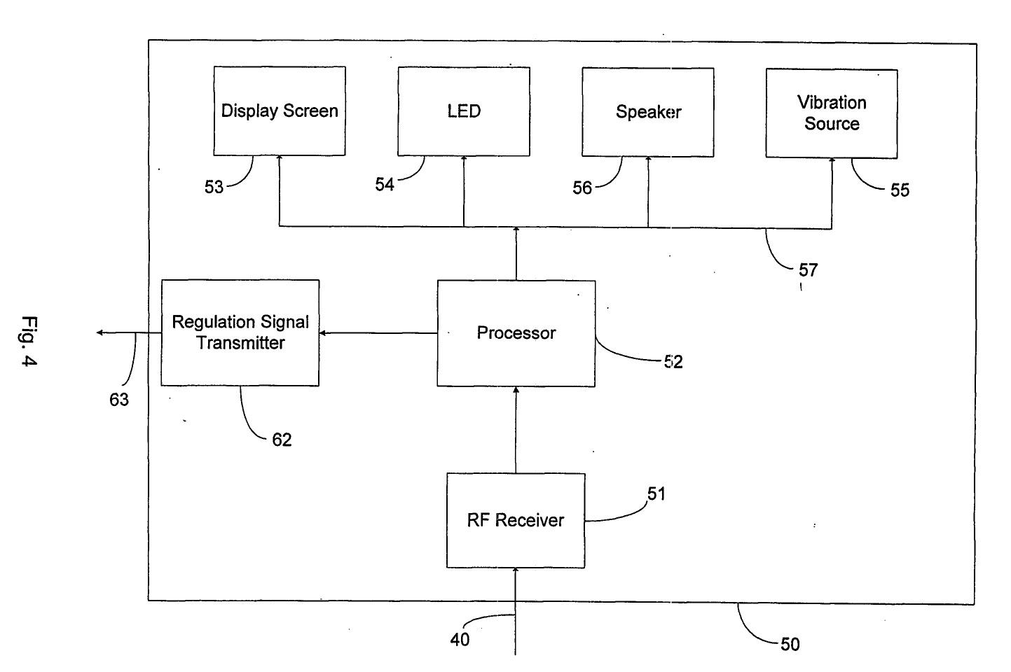

Referring now to FIG. 4, a simplified schematic of an embodiment of the circuitry within receiver 50 is illustrated. Receiver 50 comprises RF receiver 51, processor 52, display screen 53, LED 54, vibration source 55, speaker 56, and bus 57. RF receiver 51 receives RF signal 40 from solid-state manometry probe 13. RF signal 40 represents the pressure data collected from solid-state manometry probe 13. RF receiver 51 converts incoming RF signal 40 to an electrical signal and transmits this electrical signal (i.e., the pressure data) to processor 52. Processor 52 receives the pressure data measurements retrieved by solid-state manometry probe 13 and process/analyzes the pressure data to determine whether the data represents an aggravated state. This can be achieved by properly programming processor 52 in a variety of ways. For example, processor 52 can be programmed to receive pressure data from solid-state manometry probe 13 over a period of time when the bowel of patient 100 is functioning normally. Processor 52 will then recognize pressure measurements within a certain range of the normal measurements as being a non-aggravated state. Normalcy can be based upon the magnitude, frequency, spasmodic nature, or pattern of contraction pressures created in bowel 15. Alternatively, processor 52 can be pre-programmed to correlate specified pressure data parameter ranges as being non-aggravated.

Upon processor 52 processing the pressure data and detecting an “aggravated state (i.e. a state not within the range of what is considered normal bowel pressure activity), processor 52 generates and sends a warning signal to one or all of display screen 53, LED 54, speaker 56, or vibration source 55 via bus 57.

Upon receiving the warning signal, the appropriate component 53, 54, 56, 55 will respond accordingly. For example, LED 54 may be illuminated upon receiving the warning signal; speaker 56 may output a warning sound/tome, vibration source 55 may vibrate receiver 50, and/or display screen 53 may display an image. text- or pressure data relaying the aggravated state to the user. Depending on design preference, any combination of or single component 53, 54, 56, or 55 can be used to signal the aggravated state to the user.

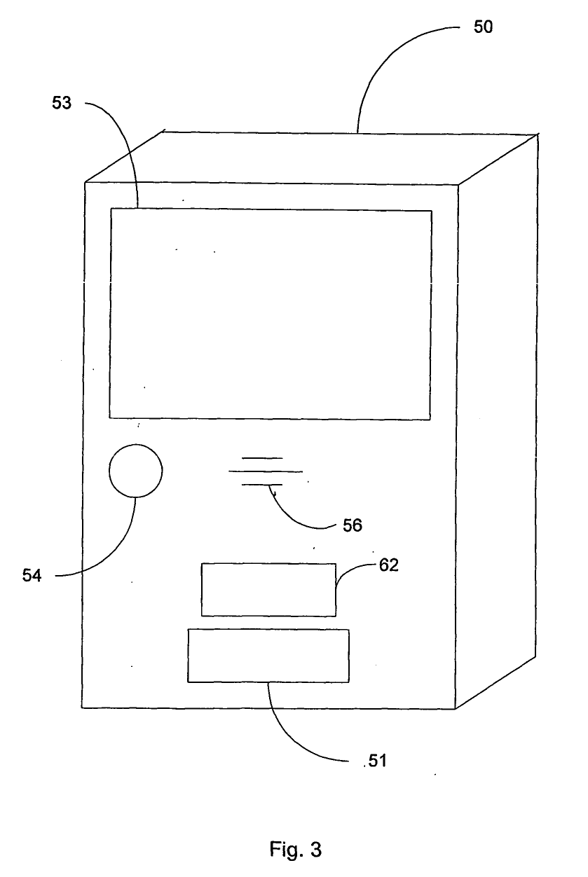

Referring now to FIG. 3, an embodiment of receiver 50 is illustrated. While illustrated as a box-like device, receiver 50 can be designed in any shape or form. For example, receiver 50 can be designed to attach to be worn around the waist of a user or can be designed to be worn like a watch. Externally, receiver 50 comprises display screen 53, LED 54, and speaker 56. Processor 52 (FIG. 4) can be programmed to display a variety of images or text messages on display screen 53 that can either warn patient 100 of the aggravated state or instruct patient 100 to take measures to alleviate, reduce, or delay onset of the symptoms related to IBS. For example, display screen 53 may instruct patient 100 to perform relaxation exercises, rest, or massage the abdomen. Moreover, processor 50 can be programmed to correlate the aggravated state to the ingestion of certain foods via dietary inputs from patient 100 or from a doctor.

It is also possible for the present invention to be used to regulate the muscle contractions of bowel wall 14 upon determination of the aggravated through the use of a colon-pace setter. Regulation of the muscle contractions of bowel wall 14 with a colon pace-setter will reestablish normalcy of frequency, magnitude of contraction, and pattern of contraction, similar to a heart pace-maker but adapted to colon physiology. Such regulation will help alleviate the aggravated state and may help IBS symptoms subside or be reduced.

The regulation aspect of the invention can be carried out using conventional pace-maker technology used in heart pace-makers, only adapted for colon/bowel physiology. An example of such technology can be found in U.S. Pat. No. 6,253,108, Rosborough, et al., which is incorporated herein by reference. The colon pace-setter is preferably incorporated into solid-state manometry probe 13. However, it is possible for the colon pace-setter to be a separate device inserted into bowel cavity 15.

Referring now to FIGS. 1 and 4, when the colon pace-setter is incorporated into state manometry probe 13, state manometry probe 13 will further comprise a regulation signal receiver 60. Regulation signal receiver 60 can be a radio frequency receiver. When the system is adapted to regulate colon muscular contractions, receiver 50 will function as described above. However, upon processor 52 determining an aggravated state, processor 52 will send an activation signal to regulation signal transmitter 62. In response to this activation signal, regulation signal transmitter 62 will generate and transmit regulation signal 63 to regulation signal receiver 60 (FIG. 2). Upon receipt of regulation signal 63, the colon pace-setter, which is incorporated into solid-state manometry probe 13, will be activated. A properly programmed processor within solid-state manometry probe 13 will control the operations of the colon pace-setter. The colon pace-setter will use electrical impulses to control/regulate the muscle contractions of bowel wall 14. The exact electrical impulses that bowel wall 14 will be subjected to by the colon pace-setter will be based on what is determined to be normal muscle activity in that patient when in a healthy non-aggravated state.

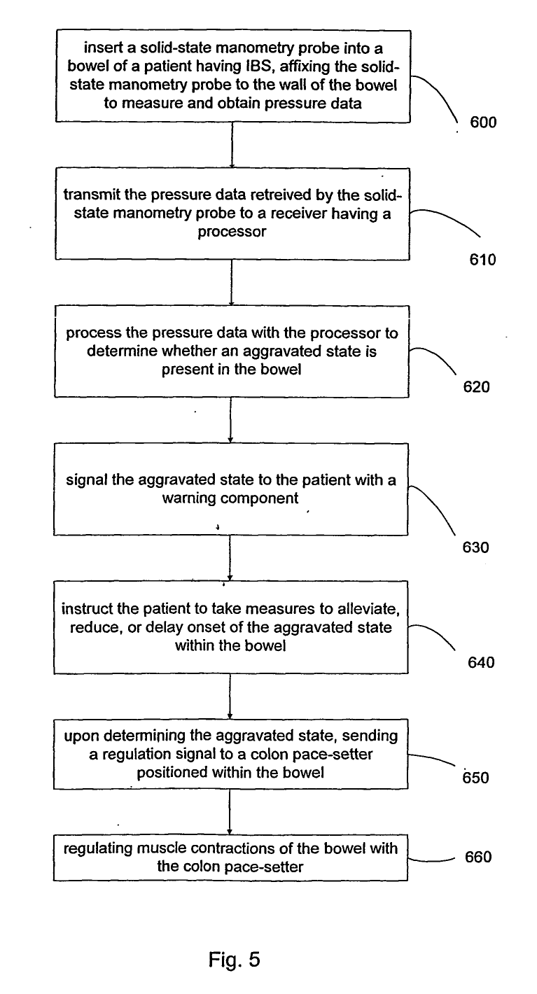

FIG. 5 is a flowchart of an embodiment of the method of using the system illustrated and described above in relation to FIGS. 1-4. In order to avoid redundancy, a detail explanation of steps 600-660 will be omitted in light of the above disclosure.

While the invention has been described and illustrated in sufficient detail that those skilled in this art can readily make and use it, various alternatives, modifications, and improvements should become readily apparent without departing from the spirit and scope of the invention. Specifically, any method of data transmission can be used to transmit the pressure data from the probe to the receiver. For example, pressure data can be transmitted to the receiver via a wire connection running through a catheter.

What is claimed is:

1. A method for treating ritable Bowel Syndrome (“IBS”) comprising:
inserting a probe into a bowel of a patient having IBS, the probe adapted to measure pressure at one more locations within the bowel and obtain pressure data;
transmitting the pressure data to a receiver having a processor;
processing the pressure data with the processor to determine an aggravated state; and
signaling the aggravated state to the patient.

2. The method of claim 1 further comprising instructing the patient to take measures to alleviate, reduce, or delay onset of the aggravated state.

3. The method of claim 2 wherein the measures are selected from the group consisting of performing relaxation exercises, resting, meditating, massaging an abdomen of the patient, and relating the aggravated state to ingestion of certain foods.

4. The method of claim 1 wherein the probe comprises a solid state manometry probe or a water perfused manometry probe.

5. The method of claim 1 wherein the pressure data obtained by the probe is transmitted to the receiver via wireless means.

6. The method of claim 5 wherein the wireless means is a radio frequency transmitter.

7. The method of claim 1 wherein the signaling step comprises creating an audio signal with the receiver, displaying an image on a display area of the receiver, illuminating a light source on the receiver, or vibrating the receiver, or displaying the measured pressure data on the receiver.

8. The method of claim 1 wherein the probe is fixed to a wall of the bowel.

9. The method of claim 1 wherein the pressure data obtained by the probe is transmitted to the receiver via a wire within a catheter.

10. The method of claim 1 further comprising instructing the patient to perform relaxation techniques to alleviate the aggravated state, wherein the pressure data obtained by the probe is transmitted to the receiver via wireless means, wherein the probe is a solid state manometry probe fixed to a wall of the bowel; and wherein the signaling step comprises creating an audio signal, displaying an image on a display area of the receiver, illuminating a light source, or vibrating the receiver.

11. The method of claim 1 further comprising:
upon determining the aggravated state, sending a regulation signal to a colon pace-setter positioned within the bowel; and
regulating muscle contractions of the bowel with the colon pace-setter.

12. A system for treating Irritable Bowel Syndrome BIBS”) comprising:
a pressure probe adapted to be inserted into a bowel of a patient having IBS, the pressure probe having means to measure pressure at one more locations within the bowel and obtain pressure data and means to transmit the pressure data;
a receiver having means to receive the pressure data transmitted by the pressure robe;
a processor programmed to process the pressure data to determine an aggravated state; and
means to signal the aggravated state to the patient.

13. The system of claim 12 further comprising means to instruct the patient to take measures to alleviate, reduce, or delay onset of the aggravated state.

14. The system of claim 13 wherein the means to instruct is an image on a display area of the receiver.

15. The system of claim 13 wherein the measures include performing relaxation exercises, resting, meditating, or avoiding foods causing increased bowel pressure.

16. The system of claim 12 wherein the means to signal is selected from a group consisting of a speaker, an image display, a light source, a vibration source, and a pressure display.

17. The system of claim 12 wherein the pressure probe comprises a solid state manometry probe or a water perfused manometry probe.

18. The system of claim 12 wherein the means to transmit the pressure data is a radio frequency transmitter and the means to receive the pressure data is a radio frequency receiver.

19. The system of claim 12 wherein the pressure probe is wireless and adapted to be fixed to a wall of the bowel.

20. The system of claim 12 wherein the means to transmit the pressure data is a wire within a catheter.

21. The system of claim 12 wherein the receiver comprises the processor and the means to signal.

22. The system of claim 12 further comprising means to instruct the patient to perform relaxation techniques to alleviate, reduce, or delay onset of the aggravated state, wherein the means to signal is selected from a group consisting of a speaker, an image display, a light source, and a vibration source, wherein the pressure probe comprises a solid state manometry probe, wherein the means to transmit the pressure data is a radio frequency transmitter and the means to receive the pressure data is a radio frequency receiver, and wherein the pressure probe is wireless and adapted to be fixed to a wall of the bowel.

23. The system of claim 12 further comprising a colon pace-setter adapted to regulate muscle contractions of the bowel upon receipt of a regulation signal, wherein the processor is adapted to generate and send the regulation signal to the colon pace-setter upon determination of the aggravated state.

24. A method for treating Irritable Bowel Syndrome (“IBS”) comprising:
inserting a probe into a bowel of a patient having IBS, the probe adapted to measure pressure at one more locations within the bowel and obtain pressure data;
inserting a colon pace-setter into the bowel, the colon pace-setter adapted to regulate muscle contractions of the bowel upon receipt of a regulation signal;
transmitting the pressure data to a receiver having a processor;
processing the pressure data with the processor to determine an aggravated state;
upon determination of an aggravated state, generating the regulation signal with the processor; and
transmitting the regulation signal to the colon pace-setter.

25. The method of claim 24 wherein the colon pace-setter and the probe are contained within a single device.

26. The method of claim 24 wherein the probe comprises a solid state manometry probe or a water perfused manometry probe.

27. The method of claim 24 wherein the pressure data obtained by the probe is transmitted to the receiver via wireless means.

28. The method of claim 27 wherein the wireless means is a radio frequency transmitter.

29. The method of claim 24 wherein the regulation signal is transmitted from the receiver to the colon-pace setter via wireless means.
